(19) United States
(12) Patent Application Publication
(10) Pub. No.: US 2020/0246601 A1
(43) Pub. Date: Aug. 6, 2020

(54) Hand-Held Fluid Substance Applicator

(71) Applicant: MATBLOK VENTURES INC., Calgary (CA)

(72) Inventors: Steven Tyler Brown, Calgary (CA); Fred Cary, San Diego, CA (US)

(21) Appl. No.: 16/837,950

(22) Filed: Apr. 1, 2020

Related U.S. Application Data
(63) Continuation of application No. 15/808,144, filed on Nov. 9, 2017.
Application No. 15/808,144 is a continuation of application No. 29/584,036, filed on Nov. 10, 2016.
(60) Provisional application No. 62/420,275, filed on Nov. 10, 2016.

(51) Int. Cl. A61M 35/00

(52) U.S. Cl. CPC A61M 35/003; A61M 2202/04

(57) ABSTRACT

A hand-held fluid substance applicator for applying a fluid substance to a skin surface is provided. The hand-held fluid substance applicator includes a first body having a middle portion extending continuously between a front side and rear side. The first body forms a first chamber that can hold a fluid substance, wherein the first chamber is in fluid communication with a first pad. A first valve dispenses the fluid substance to the first pad upon increased pressure in the first chamber. A second body is removably secured to the first body and includes a second chamber in fluid communication with a second pad, wherein the second chamber can hold the fluid substance. The first pad is used to apply the fluid substance to a first part of a user's body and the second pad is used to apply the fluid substance, or a second fluid substance, to a second part of a user's body.

### CROSS REFERENCE TO RELATED APPLICATION

This application claims the benefit of U.S. Provisional Application No. 62/420,275 filed on Nov. 10, 2016 and U.S. Nonprovisional application Ser. No. 15/808,144 filed on Nov. 9, 2017. The above identified patent applications are herein incorporated by reference in their entirety.

### BACKGROUND OF THE INVENTION

The present invention relates to a hand-held fluid applicator. More specifically, the present invention relates to a hand-held fluid applicator having a first chamber removably secured to a second chamber, wherein each chamber comprises an opening and a pad disposed thereover. The pad is configured to apply a fluid substance held within each chamber directly to a skin's surface of a user.

Applicators are typically used for applying fluid substances to a person's skin surface in order to avoid direct contact with a user's hand. Hand-held fluid substance applicators for fluids, such as sunscreen, moisturizer, makeup, insect repellant, and the like, are designed to reduce bacterial contamination and mess associated with applying these fluids by hand. Further, there is a constant demand for convenient devices that allow the application of topical skin products due to the need to protect people from skin diseases and injuries, such as sunburns and skin cancer.

Existing fluid applicators discharge fluid in a manner which has disadvantages such as discharging inconsistently and creating a mess. Moreover, these known applicators waste the product by requiring second applications of the product and over saturation during periods of use and non-use. For example, some applicators for dispensing sunscreen or insect repellant, utilize a container that holds a fluid substance and a propellant to emit an aerosol spray. However, the aerosol spray is not well suited for applying the fluid substance to selective areas of a person's skin surface. Aerosol dispersed fluid substances tend to be dispersed over a relatively wide area.

Some other known types of applicators for dispensing a fluid substance utilize a roller ball mechanism. However, such devices are prone to leaving visible streaks/tracks of the fluid substance on a person's skin. Additionally, such devices are not well suited to applying a fluid substance to a broad surface area in a reasonable time due to the relatively small applicator surface area that contact's a person's skin surface.

Further, existing fluid applicators only have a single mechanism for discharging a single substance. Thus, a person will likely not want to apply a moisturizer to his or her face using the same applicator used for his or her back or legs. Additionally, different regions of the body and face may require a different fluid for achieving a same purpose. For example, many face moisturizers are not intended for use on the body and vice versa. Therefore, there is a need in the art for a hand-held fluid substance applicator that comprises a first chamber removable secured to a second chamber that is configured to apply fluid to different parts of a user's body and face.

In light of the devices disclosed in the known art, it is submitted that the present invention substantially diverges in design elements and methods from the known art and consequently it is clear that there is a need in the art for an improvement for a hand-held fluid substance applicator and method of use. In this regard the instant invention substantially fulfills these needs.

### SUMMARY OF THE INVENTION

In view of the foregoing disadvantages inherent in the known types of hand-held fluid substance applicators now present in the known art. The present invention provides a new handheld fluid applicator, wherein the same can be utilized for applying a fluid substance directly to a skin's surface.

It is an objective of the present invention to provide an embodiment of the hand-held fluid substance applicator comprising a first body including a front side, a rear side, and a middle portion, wherein the middle portion extends continuously between the front and rear sides, respectively. The first body forms a first chamber configured to hold a fluid substance, wherein the first chamber is in fluid communication with a first pad via a first valve. The first valve is configured to dispense the fluid substance from the first chamber to the first pad upon increased pressure in the first chamber.

It is another objective of the present invention to provide an embodiment of the hand-held fluid substance applicator wherein the middle portion is deformable such that compression of the first body causes a pressure transfer within the first chamber which in turn causes the first valve to dispense the fluid substance during fluid application.

It is another objective of the present invention to provide an embodiment of the hand-held fluid substance applicator comprising a second body removably secured to the first body, the second body having a second chamber configured to hold the fluid substance. A second pad is in fluid communication with the second body via a second valve, wherein the second valve is configured to release the fluid substance from the second chamber via gravity.

It is another objective of the present invention to provide an embodiment of the hand-held fluid substance applicator comprising an applicator arm pivotally secured to the first body for applying the fluid substance onto difficult to reach places of a user's body.

It is therefore an object of the present invention to provide a new and improved hand-held fluid applicator that has all of the advantages of the known art and none of the disadvantages.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front perspective view of a first embodiment of the hand-held fluid substance applicator.

FIG. 2 shows a side view of a first embodiment of the hand-held fluid substance applicator.

FIG. 3 shows an exploded view of a first embodiment of the hand-held fluid substance applicator.

FIG. 4 shows a rear perspective view of a first embodiment of the hand-held fluid substance applicator wherein the applicator arm is in a raised position.

FIG. 5A shows a cross sectional view of a first embodiment of the hand-held fluid substance applicator of FIG. 1.

FIG. 5B shows a close up view of the first valve of a first embodiment of the hand-held fluid substance applicator.

FIG. 6 shows a front perspective view of a second embodiment of the hand-held fluid substance applicator.

FIG. 7 shows an exploded view of a second embodiment of the hand-held fluid substance applicator.

FIG. 8 shows a rear perspective view of a second embodiment of the hand-held fluid substance applicator wherein the applicator arm is in a raised position.

FIG. 9 shows a side view of a third embodiment of the hand-held fluid substance applicator.

FIG. 10 shows a front perspective view of a third embodiment of the hand-held fluid substance applicator.

FIG. 11 shows a perspective view of a third embodiment of the hand-held fluid substance applicator in use.

### DETAILED DESCRIPTION OF THE INVENTION

Reference is made herein to the attached drawings. Like reference numerals are used throughout the drawings to depict like or similar elements of the hand-held fluid substance applicator. For the purposes of presenting a brief and clear description of the present invention, the preferred embodiment will be discussed as used for applying a fluid substance to a user. The figures are intended for representative purposes only and should not be considered to be limiting in any respect. Furthermore, the described features, structures, or characteristics may be combined in any suitable manner in one or more embodiments. In the following description, numerous specific details are provided to give a thorough understanding of embodiments.

Reference will now be made in detail to the exemplary embodiment (s) of the invention. References to “one embodiment,” “at least one embodiment,” “an embodiment,” “one example,” “an example,” “for example,” and so on indicate that the embodiment(s) or example(s) may include a feature, structure, characteristic, property, element, or limitation but that not every embodiment or example necessarily includes that feature, structure, characteristic, property, element, or limitation. Further, repeated use of the phrase “in an embodiment”, “first embodiment”, “second embodiment”, or “third embodiment” does not necessarily refer to the same embodiment.

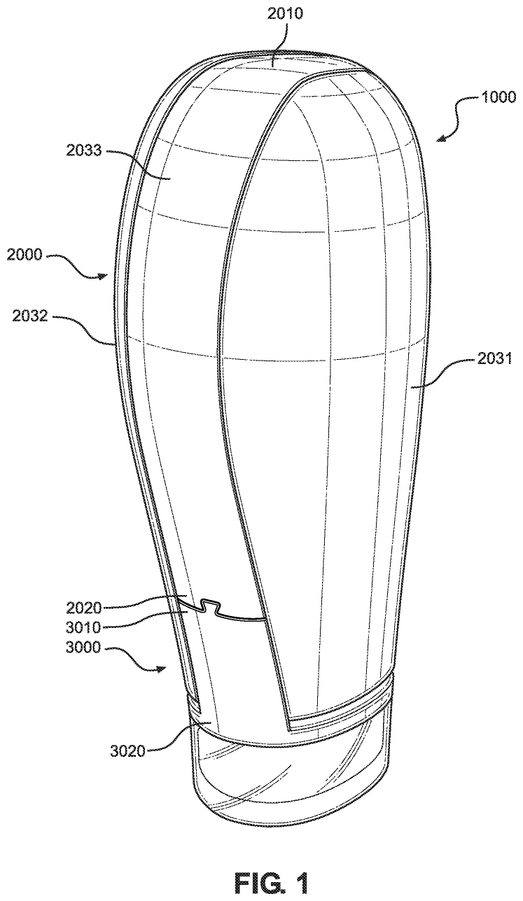

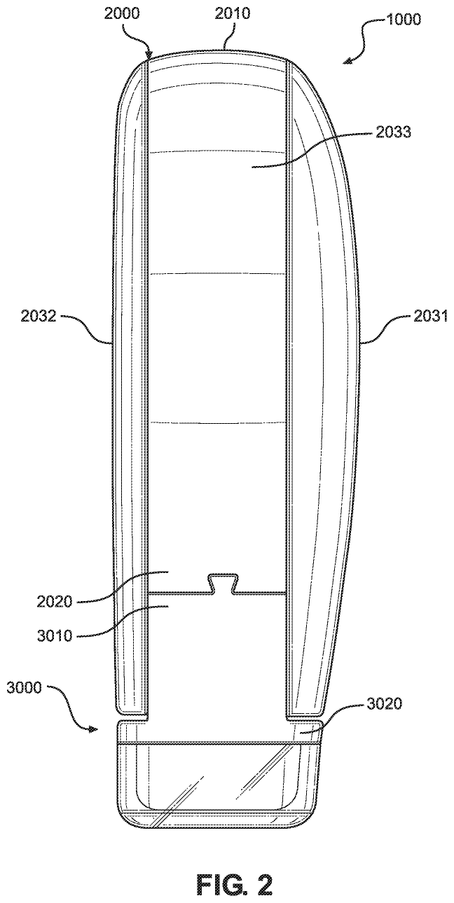

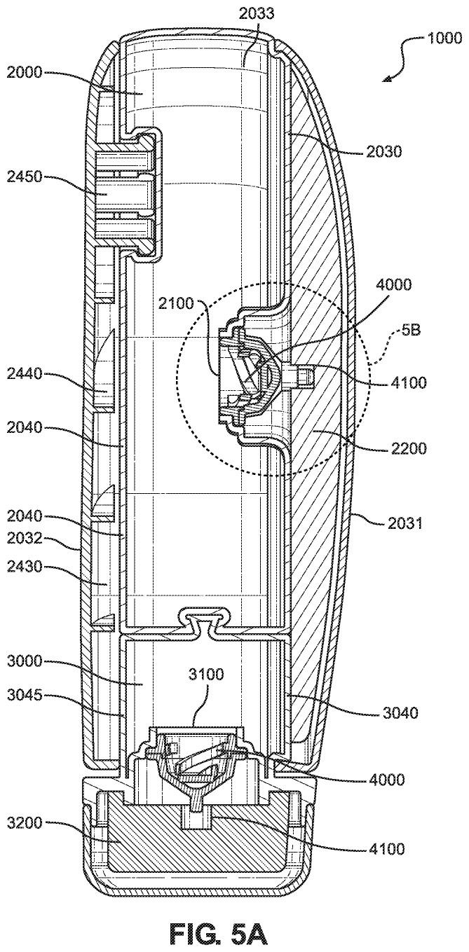

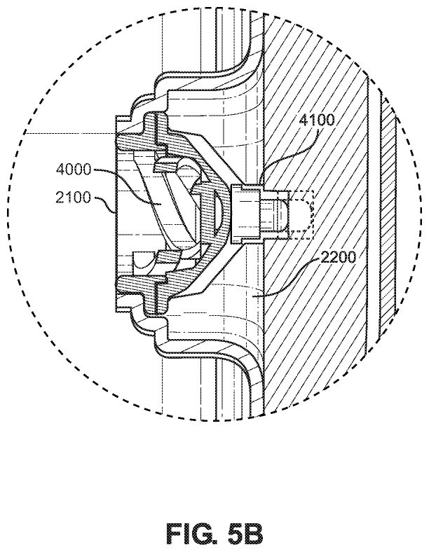

Referring now to FIGS. 1 and 2, there is shown a front perspective view and a side view of a first embodiment of the hand-held fluid substance applicator, respectively. The hand-fluid applicator 1000 comprises a first body 2000 having a front side 2031, a rear side 2032, and a middle portion 2032. In the illustrated embodiment, the middle portion 2033 extends continuously between the front and rear sides 2031, 2032, respectively. The first body 2000 forms a first chamber (as shown in FIG. 5) configured to hold a fluid substance, such as sunscreen, insect repellant, makeup, or the like. The first chamber 2000 is in fluid communication with a first pad, wherein the first pad is configured to apply the fluid substance directly to a skin surface, such as the arms, back, torso, and legs of the user.

The illustrated embodiment comprises a second body 3000 removably secured to the first body 2000 and includes a second chamber (as shown in FIG. 5) that can hold the fluid substance. The second chamber is in fluid communication with a second pad. The second pad is used to apply the fluid substance to a different part of a user's body than the first pad, such as the face of the user. In this way, cross contamination between the first skin surface and the second skin surface is prevented through the use of distinct pads. Parts of the body produce and accumulate different bacteria than other parts of the body. For example, oils and dirt accumulate on the hands and arms of a user that the user will not want to transfer to his or her face. Further, in the illustrated embodiment, a first fluid substance within the first chamber can be different than a second fluid substance in the second chamber to allow a user to apply a fluid intended for the body to be disposed in the first body 2000 and another fluid intended to the be applied to the face to be disposed in the second body 3000.

In the illustrated embodiment, the first body 2000 and second body 3000 are disposed in a stacked configuration, such that the first body 2000 is disposed above the second body 3000 when the hand-held fluid substance applicator 1000 is in an upright position. The first body 2000 comprises a first end 2010 and an opposing second end 2020, wherein a first end 3010 of the second body 3000 is secured to the second end 2020 of the first body 2000. In alternate embodiments, the first and second bodies are disposed in a side-by-side configuration, such that both the first and second bodies are configured to rest directly on a support surface.

In the illustrated embodiment, the first end 2010 of the first body 2000 is bulbous, such that the width of the first body 2000 tapers towards the second end 2020 thereof. The bulbous shape of the first body 2000 allows the hand-held fluid substance applicator 1000 to have a lower center of gravity in the upright position, providing more stability thereto. The upright position is defined as the first body 2000 resting above the second body 3000 and a second end 3020 of the second body 3000 positioned closest to a support surface.

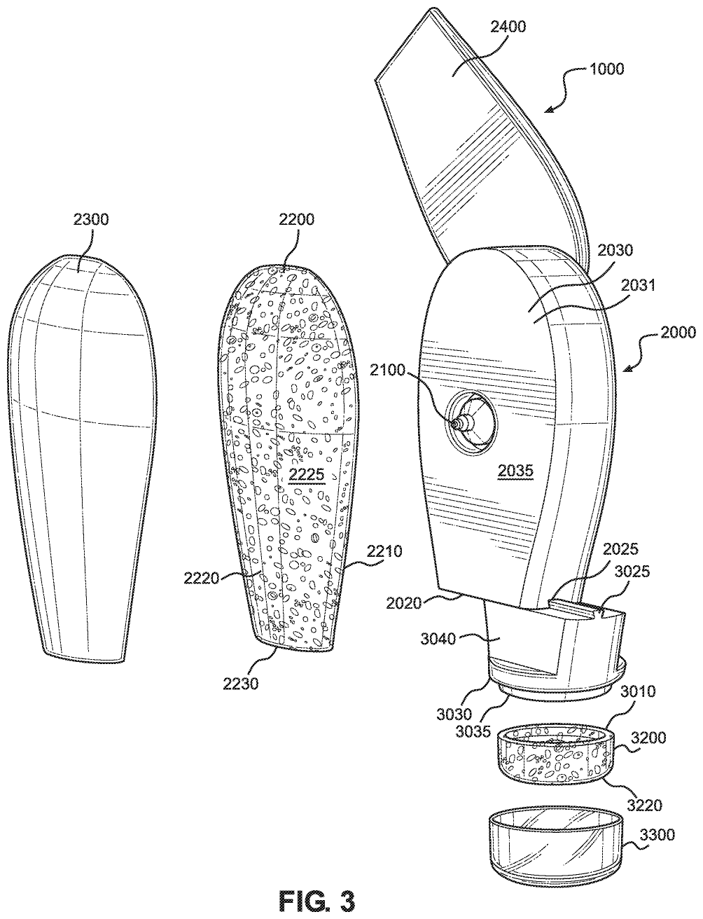

Referring not to FIG. 3, there is shown an exploded view of a first embodiment of the hand-held fluid substance applicator. The first chamber is in fluid communication with the first pad 2200 via a first valve 2100. The first valve 2100 dispenses the fluid substance to the first pad 2200 upon increased pressure in the first chamber. The first valve 2100 extends through a sidewall 2030 of the front side 2031 of the first body 2000. In the illustrated embodiment, the first valve 2100 is disposed centrally on the sidewall 2030 to distribute the fluid substance released from the first chamber on a central area of a first pad 2200. In the illustrated embodiment, the first body 2000 comprises a single first valve 2100. However, in alternate embodiments, the first body 2000 comprises a plurality of first valves distributed along the sidewall thereof.

The first pad 2200 comprises a first side 2210 and an opposing second side 2220, wherein the first side 2210 is flat and rests flush against the sidewall 2030 of the first body 2000. The second side 2220 is configured for contacting a skin surface. The second side 2220 of the first pad 2200 is contoured in order to provide better contact between the pad 2200 and the skin surface. In the illustrated embodiment, a surface 2225 of the second side 2220 comprises a convex curve that tapers towards a perimeter thereof.

In the illustrated embodiment, a lower end 2230 of the first pad 2200 extends beyond the sidewall 2030 of the first body 2000 and is disposed on the sidewall 3040 of the second body 3000. In alternate embodiments, the lower end of the first pad 2200 terminates at a lower end of the sidewall 2030 of the first body 2000. In the illustrated embodiment, the lower end 2230 of the first pad 2200 comprises a linear edge configured to rest above a base 3030 of the second body 3000. The base 3030 extends outward from the sidewalls 2040, 3040 to serve as a shoulder for a first body cap 2300.

In the illustrated embodiment, the first body cap 2300 is disposed over the first pad 2200 and removably secures to the first body 2000. The shape of the first pad 2200 corresponds to the shape of the sidewall 2030 and substantially covers the surface 2035 of sidewall 2030 without extending beyond a perimeter of the sidewall 2030. In this way, the first body cap 2300 can enclose the first pad 2200 while simultaneously having a perimeter that aligns with the perimeter of the sidewall 2030 of the first body 2000. In some embodiments, the first body cap 2300 comprises a slightly larger tolerance than the perimeter of the front side 2031 of the first body 2000 such that the cap 2300 removably secures thereto via a friction fit. In alternate embodiments, the first body cap 2300 removably secures over the first pad 2200 in any suitable manner such that the first body cap 2300 is removably securable to the first body 2000. The first body cap 2300 comprises an interior recess having a same shape as the first pad 2200 to receive the first pad 2200 therein without excess space therebetween.

The second chamber is in fluid communication with the second pad 3200 via a second valve (FIG. 5, 3100) at a base 3030 thereof. The second valve is disposed centrally on the base 3030 to distribute the fluid substance released from the second body 3000 on a central area of a second pad 3200. The second pad 3200 comprises a first side 3210 and an opposing second side 3220, wherein the first side 3210 faces the base 3030 and the second side 3220 is configured for contacting the skin surface. In the illustrated embodiment, a front face of the first pad 2200 is oriented perpendicular to a front face of the second pad 3200.

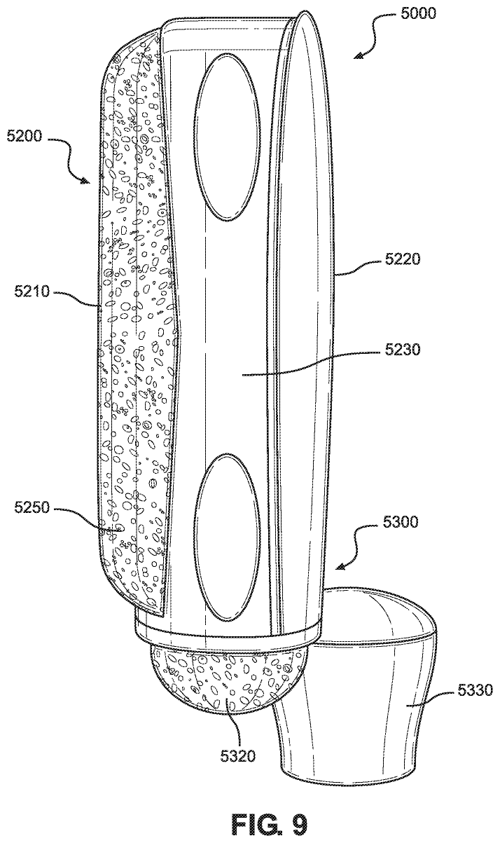

The cross-sectional shape of the second pad 3200 is circular and corresponds to the shape of the base 3030 such that the second pad 3200 substantially covers the surface of the base 3030. The base 3030 comprises an interior lip 3035 designed to fit within a recess of the second pad 3200. In this way, the first side 3210 of the second pad 3200 is configured to rest flush against the base 3030, whereas the lip 3035 provides additional support for the pad secured thereto. The second side 3220 of the second pad 3200 is flat. However, in alternated embodiments, a surface of the second side 3220 is curved in order to provide a contoured contact between the second pad 3200 and the skin surface (as seen in FIG. 9).

In the illustrated embodiment, a second body cap 3300 is disposed over the second pad 3200 and removably secures to the second body 3000. When secured to the second body 3000, the second body cap 3300 is disposed around the lip 3035 and comprises a same outer diameter of the base 3030 so as to remain flush along the exterior therewith and create a continuous exterior surface. In some embodiments, the second body cap 3300 comprises a slightly larger tolerance than the perimeter of the second body 3000 such that the cap 3300 removably secures thereto via a friction fit. In alternate embodiments, the second body cap 3300 removably secures over the second pad 3200 in any suitable manner such that the second body cap 3300 is removably securable to the second body 3000. In the illustrated embodiment, the second body cap 3300 comprises an interior recess having a same shape as the second pad 3200 to receive the second pad 3200 therein without excess space therebetween.

In the shown embodiment, the first body 2000 is removably secured to the second chamber 3000 via a mating fastener. The second end 2020 of the first body 2000 comprises a recess 2025 that slidably receives a tab 3035 disposed on the first end 3010 of the second body 3000, wherein the recess 2025 and the tab 3025 comprise a corresponding cross-sectional shape. In the shown embodiment, the shape is dovetail wherein the sidewalls of the recess and tab taper inwards to prevent the first body 2000 from inadvertently detaching from the second body 3000 in a motion other than a sliding motion along a horizontal axis.

The recess 2025 and tab 3025 extend entirely along the horizontal axis of the middle portions of both the first and second chambers 2000, 3000, respectively. In this way, additional stability is provided thereto. In some embodiments, a flange is positioned along an end of the recess in order to prevent the second body from sliding into a first lateral side of the first body and beyond an opposing second lateral side thereof. The lower most face of the first body 2000 and the upper most face of the second body 3000 are flat in order to rest flush against one another when attached together.

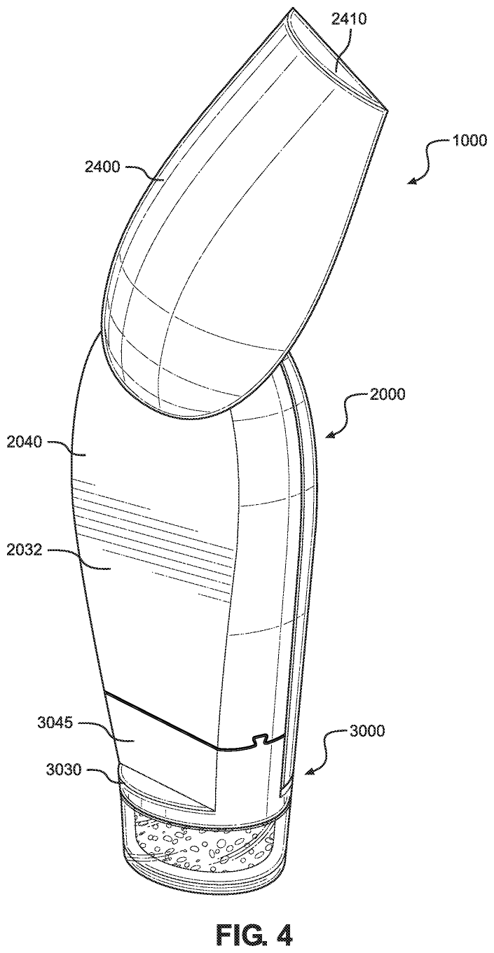

Referring now to FIG. 4, there is shown a perspective view of a first embodiment of the hand-held fluid substance applicator wherein the applicator arm is in a raised position. In some embodiments, an applicator arm 2400 is pivotally secured to the rear side 2032 of the first body and configured to assist a user in applying or spreading the fluid substance the skin surface. In use, once fluid is applied to the skin surface, such as the back of the user, the applicator arm 2400 can be pivoted to a raised position. The applicator arm 2400 receives the fluid substance, wherein the user then grasps the first body 2000 and positions the applicator arm against their back. The applicator arm 2400 is used to spread the fluid substance to lower portions of the back that the user would be unable to reach otherwise.

In the shown embodiment, the applicator arm 2400 comprises a similar shape as the first body cap, wherein the exterior surface of the applicator arm 2400 is contoured. An interior surface of the applicator arm 2400 is flat and configured to rest flush against a sidewall 2040 of the rear side 2032 of the first body 2000. In some embodiments, the applicator arm 2400 further extends over a sidewall 3045 of the rear side 2032 of the second body 3000.

A lower end 2410 of the applicator arm 2400 is flat and configured to rest above or flush against the base 3030 of the second body 3000 in order to form a continuous exterior surface. In the way, the base 3030 and the rear side of the second body 3000 forms a shoulder to receive the applicator arm 2400 when in a closed position. However, in alternate embodiments, the hand-held fluid substance applicator 1000 does not comprise an applicator arm. In some embodiments, the rear side of the second body 2000 forms an exterior to the hand-held fluid substance applicator 1000, wherein the applicator arm is not included.

The applicator arm 2400 comprises a hollow interior (FIG. 5, 2430) having a plurality of braces (FIG. 5, 2440) extending therethrough for providing integral support. The hollow interior provides a lighter weight hand-held fluid substance applicator 1000 and prevents excess weight or stress on the pivot joint (FIG. 5, 2450). In some embodiments, the applicator arm is solid. In other embodiments, the applicator arm is removably or slidably secured to the rear side of the first body.

Referring now to FIGS. 5A and 5B, there is shown a cross sectional view of a first embodiment of the hand-held fluid substance applicator of FIG. 1 and a close up view of the first valve of a first embodiment of the hand-held fluid substance applicator, respectively. The first and second bodies 2000, 3000 are each configured to release a fluid substance through the first and second valves 2100, 3100 and into the first and second pads 2200, 3200, respectively. In the illustrated embodiment, the second chamber is configured to release fluid through the second valve when an operator exerts a force on the second chamber 3001, causing a positive pressure thereto, for the valve is configured to control the flow of the fluid substance through the second chamber 3001. When the operator exerts force by squeezing the second chamber, the valve opens, and fluid is released through an opening and into the pad. Once the fluid is disposed on the second pad, the operator rubs the pad on the skin surface, thereby spreading the fluid around onto the targeted skin surface and effectively integrating the fluid substance until it is absorbed by the skin. In alternate embodiments, the second valve is configured to release fluid to the second pad from the second chamber via gravity.

In the illustrated embodiment, the first and second valves 2100, 3100 each comprise a protrusion 4100 that extends beyond the sidewall of the first body and the base of the second chamber and into the respective pads. In this way, the protrusion delivers the fluid substance deeper within the pad. In the shown embodiment, the protrusion 4100 of the first valve 2100 is depressible toward an interior of the first chamber 2001 such that when the protrusion is depressed, fluid is released from the first chamber into the first pad. In operation, a user presses the first pad against his or her skin. As the pad contacts the user causing force against the first valve, the protrusion will depress toward the interior of the first chamber thereby releasing fluid therefrom.

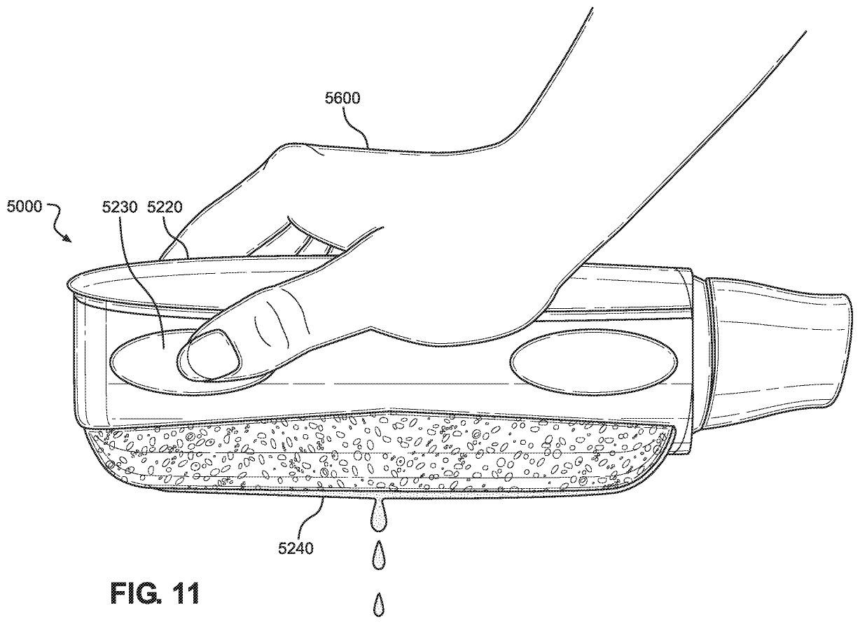

In alternate embodiments, the first chamber is configured to release fluid through the first valve when an operator exerts a force on the first chamber, causing a positive pressure thereto, as seen in FIG. 11. When the operator exerts force by squeezing opposing sidewalls of the first chamber, the valve opens, and fluid is released through an opening and into the pad. In some embodiments, the first valve is configured to release fluid to the first pad from the first chamber via gravity.

In the illustrated embodiment, the first chamber 2001 and second chamber 3001 comprise an equal width, wherein the width is measured between the sidewalls of the front and rear sides thereof. The first chamber 2001 is distinct from the second chamber 3001, meaning that fluid does not pass between the chambers. However, in alternate embodiments, the first chamber is in fluid communication with the second chamber via a shared opening.

In the illustrated embodiment, the second chamber comprises a smaller interior volume than the first chamber, such that the first chamber is configured to hold a larger volume of fluid substance. In this way, the fluid substance in the first chamber 2000 will deplete at a same rate as the fluid substance in the second chamber 3000 since the first chamber is intended for use on the body and the second chamber is intended for use on the face.

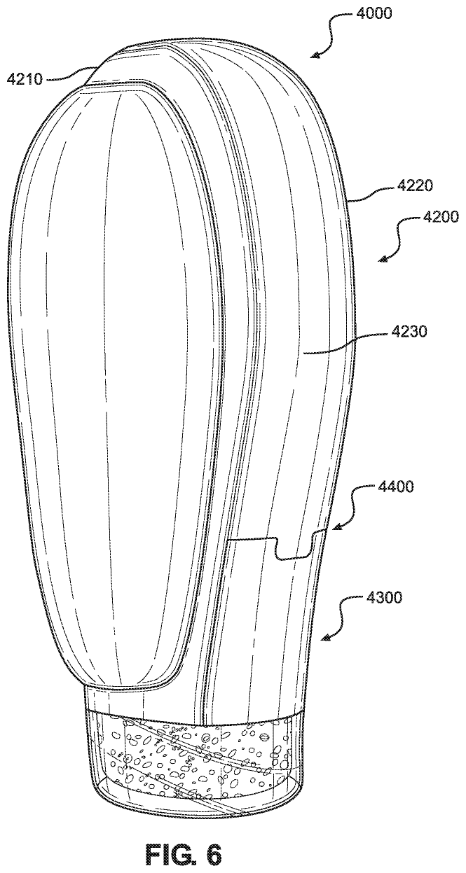

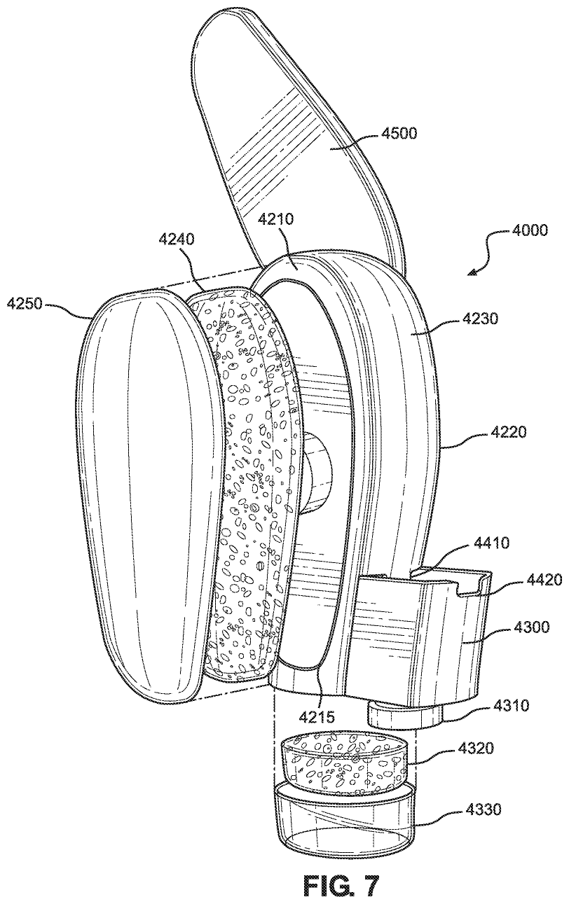

Referring now to FIGS. 6 and 7, there is shown front perspective view and an exploded view of a second embodiment of the hand-held fluid substance applicator, respectively. In the second embodiment, the hand-held fluid substance applicator comprises a first body 4200 and second body 4300. However, the second body 4300 is removably disposed on an interior of the middle portion 4230 of the first body 4200 and between the front and rear sides 4210, 4220 thereof. The second body 4300 is disposed at a lower end of the of the first body 4200 and comprises an upper opening configured to receive the fluid substance therein. The upper opening is accessible to a user when the second body 4300 is removed from the interior of the first body via mating fasteners 4400. In the illustrated embodiment, the mating fasteners 4400 are a recess 4420 disposed on an upper edge of the second body 4300 and a corresponding tab extending from the middle portion 4230 of the first body 4200. When the recess 4420 is aligned with and receives the tab 4230 therein, the second body 4300 is completely disposed within the first body 4200.

In the illustrated second embodiment, the front side 4210 of the first body 4200 comprises a groove 4215 that receives the perimeter of the second body cap 4250, such that a perimeter of the front side 4210 extends beyond a perimeter of the first body cap 4250. The first pad 4240 is disposed interior to the groove 4215.

A bottom of the first body is open in order to allow the lip 4310 of the second body 4300 to extend therethrough. The lip 4310 receives the second pad 4320 as described in the discussion of the first embodiment. The second body cap 4330 is directly securable to the bottom of the first body 4200.

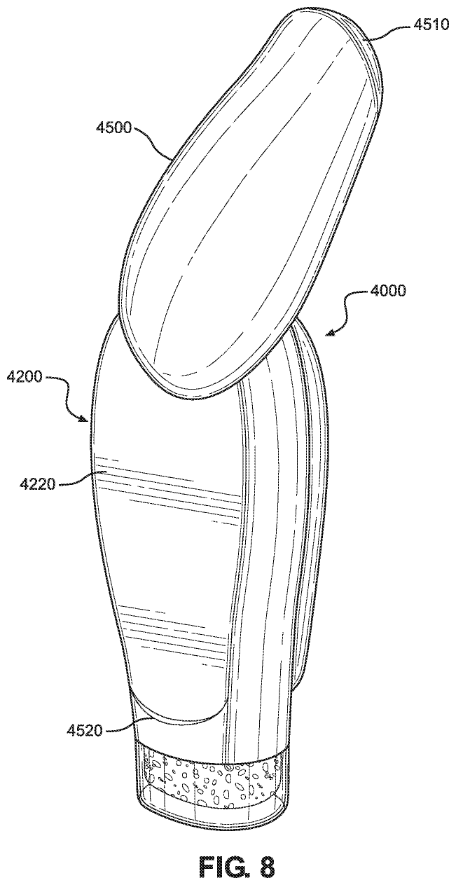

Referring now to FIG. 8, there is shown a rear perspective view of a second embodiment of the hand-held fluid substance applicator wherein an applicator arm is in a raised position. In the second embodiment, the rear side 4220 of the first body 4200 comprises a shoulder 4520 protruding from a sidewall thereof. The shoulder 4520 is curved and extends between a pair of opposing lateral sides of the first body 4200. The shoulder is configured to receive a curved lower end of the applicator arm 4500 when in a stored position. In the stored position, the applicator arm 4500 forms a continuous surface with the shoulder 4520.

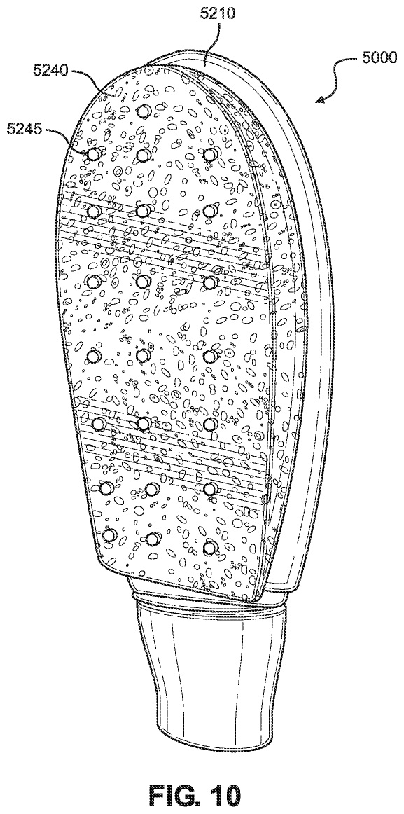

Referring now to FIGS. 9 and 10, there is shown a side view and a front view of a third embodiment of the hand-held fluid substance applicator. In the third embodiment, the second body 5300 is disposed within the first body 5200 and not separable therefrom. In some embodiments, the second body comprises a distinct second chamber from the first chamber. However, in other embodiments, the first and second body 5200, 5300 share a single chamber.

In the shown embodiment, the first pad 5250 is disposed on the front side 5210 and comprises a plurality of raised members 5245 configured to assist a user when rubbing or otherwise integrating the fluid substance onto the body. The raised members 5245 comprises a same material of the first pad and are disposed along the surface of the first pad 5250 at a fixed distance from one another. In the illustrated embodiment, the second pad 5320 is rounded in order to provide better surface contact between the pad and the user. The second body cap 5330 is non-transparent and configured to support the hand-held fluid substance applicator 5000 in an upright position.

Referring now to FIG. 11, there is shown a perspective view of a third embodiment of the hand-held fluid substance applicator in use. In some embodiments, the middle portion 5230 comprises indicia configured to provide indicate to a user how to apply pressure to the first chamber to release the fluid therefrom. In the illustrated embodiment, the indicia is a raised surface to show finger and hand placement. The middle portion is deformable such that compression of the first body causes a pressure transfer within the first chamber which in turn causes the first valve to dispense the fluid substance during fluid application. In operation, the first chamber is positioned horizontally such that the front face of the first pad is facing a ground surface. When a user places his or her hands around the middle portion of the first body and squeezes on either side thereof, the pressure is increased within the first chamber and the valve opens to release the fluid substance therefrom. The horizontal positioning of the first body allows the fluid substance to evenly distribute and saturate the first pad. In order to dispense the fluid substance from the second chamber, a user repositions the first body vertically, such that the hand-held fluid substance applicator is in the upright position and allows gravity to dispense the fluid substance to the second pad.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A hand-held fluid substance applicator, comprising:
a first body including a front side, a rear side, and a middle portion, wherein the middle portion extends continuously between the front and rear sides, respectively;
wherein the first body forms a first chamber configured to hold a fluid substance, wherein the first chamber is in fluid communication with a first pad via a first valve;
wherein the first valve is configured to dispense the fluid substance from the first chamber to the first pad upon increased pressure in the first chamber;
wherein the middle portion is deformable such that compression of the first body causes a pressure transfer within the first chamber which in turn causes the first valve to dispense the fluid substance during fluid application.

2. The hand-held fluid substance applicator of claim 1, further comprising a second body removably secured to the first body, the second body having a second chamber configured to hold a second fluid substance.

3. The hand-held fluid substance applicator of claim 2, further comprising a second pad in fluid communication with the second body via a second valve, wherein the second valve is configured to release the second fluid substance to the second pad from the second chamber via gravity.

4. The hand-held fluid substance applicator of claim 2, further comprising a second pad in fluid communication with the second body via a second valve, wherein the second valve is configured to release the second fluid substance to the second pad from the second chamber when compressed.

5. The hand-held fluid substance applicator of claim 2, wherein the first body and the second body form a continuous surface when secured together via mating fasteners.

6. The hand-held fluid substance applicator of claim 5, wherein the first pad is secured to a front face of both the first body and second body.

7. The hand-held fluid substance applicator of claim 2, wherein the second chamber comprises a smaller interior volume than the first chamber.

8. The hand-held fluid substance applicator of claim 1, wherein the first valve protrudes from the front side of the first body and is received within a recess of the first pad for saturation of the first pad.

9. The hand-held fluid substance applicator of claim 1, further comprising an applicator arm pivotally secured to the first body.

10. The hand-held fluid substance applicator of claim 9, wherein the applicator arm is disposed on the rear side of the first body and comprises a same width of the first body.

11. The hand-held fluid substance applicator of claim 9, wherein the applicator arm comprises a flat interior surface configured to rest flush against a sidewall of the first body.

12. The hand-held fluid substance applicator of claim 9, wherein the applicator arm comprises a flat interior surface configured to rest flush against a sidewall of the first body.

13. The hand-held fluid substance applicator of claim 9, wherein the applicator arm comprises a curved exterior surface that tapers from an upper end to a lower end thereof.

14. The hand-held fluid substance applicator of claim 9, further comprising a second body removably secured to the first body, the second body having a second chamber configured to hold a second fluid substance, wherein the second body comprises base that extends beyond a sidewall thereof, wherein the base and sidewall form a shoulder configured to receive a lower end of the applicator arm, such that when the applicator arm is in a closed position the applicator arm and the base form a continuous outer surface and rest flush against one another.

15. The hand-held fluid substance applicator of claim 2, wherein a second end of the first body comprises a recess that slidably receives a tab disposed on a first end of the second body, wherein the recess and tab comprise a corresponding cross-sectional shape.

16. The hand-held fluid substance applicator of claim 1, wherein the first pad comprises a bulbous first end that tapers inward toward a second end having a linear edge.

17. The hand-held fluid substance applicator of claim 2, wherein a front face of the first pad is oriented perpendicular to a front face of a second pad.

18. The hand-held fluid substance applicator of claim 2, further comprising a first body cap configured to removably enclose the first pad and a second body cap configured to removably enclose a second pad.

19. The hand-held fluid substance applicator of claim 18, wherein the second cap is configured to support the hand-held fluid substance applicator in an upright position on a surface.

20. The hand-held fluid substance applicator of claim 1, wherein the first valve comprises a depressible protrusion extending into an interior of the first pad, such that when the protrusion is depressed via force applied to the first pad fluid is released from the first chamber into the first pad.
